(19) United States
(12) Patent Application Publication
(10) Pub. No.: US 2004/0146945 A1
(43) Pub. Date: Jul. 29, 2004

(54) Olfactory neuron cultures and method of making and using the same

(75) Inventors: George Perry, University Heights, OH (US); Mark A. Smith, Cleveland, OH (US); Hossein A. Ghanbari, Potomac, MD (US)

Correspondence Address: EDWARDS & ANGELL, LLP, P.O. BOX 55874, BOSTON, MA, 02205, US

(73) Assignee: Panacea Pharmaceuticals, Inc.

(21) Appl. No.: 10/422,005

(22) Filed: Apr. 23, 2003

Related U.S. Application Data
(62) Division of application No. 09/901,186, filed on Jul. 9, 2001.
(60) Provisional application No. 60/216,648, filed on Jul. 7, 2000.
(60) Provisional application No. 60/217,087, filed on Jul. 10, 2000.

(51) Int. Cl. G01N 33/53

(52) U.S. Cl. 435/7.2

(57) ABSTRACT

Olfactory neuron cultures and methods of making and using the same are disclosed. The olfactory neuron cultures may be used to study oxidative stress-related disorders and diseases such as Alzheimer's disease. The olfactory neuron cultures may be used to screen candidate compounds for those which reduce, inhibit or prevent oxidative stress or damage. The compounds which reduce, inhibit or prevent oxidative stress or damage may be used to treat Alzheimer's disease and other oxidative stress-related disorders and diseases.

### FIELD OF THE INVENTION

The present invention relates to olfactory neuron cultures and methods of making and using thereof for the study and treatment of oxidative stress related disorders and diseases such as Alzheimer's.

### BACKGROUND OF THE INVENTION

Alzheimer's disease, the leading cause of senile dementia, is characterized pathologically by regionalized neuronal death and an accumulation of intraneuronal and extracellular lesions commonly known as neurofibrillary tangles and senile plaques, respectively. See Smith (1998) Alzheimer's Disease. In International Review of Neurobiology (Bradley, R. J. and Harris, R. A., eds.), Vol42, pp. 1-54, Academic Press, San Diego. A number of hypotheses link these pathological changes with, among others, apolipoprotein E genotype, phosphorylation of cytoskeletal proteins, and amyloid-β metabolism. See Corder, et al. (1993) Science 261:921-923; Roses (1995). Exp Neurol 132:149-156; Trojanowski, et al. (1993) Clin. Neurosci. 1:184-191; and Selkoe (1997) Science 275:630-631.

These theories, however, are insufficient to explain the spectrum of abnormalities found in Alzheimer's disease. Additionally, perturbation of these elements in cell or animal models do not result in the same multitude of biochemical and cellular changes. For example, in transgenic rodent models over-expressing β-protein precursor, where amyloid-β plaques are deposited, there is no neuronal loss. See Irizarry, et al. (1997) J. Neuropathol. Exp. Neurol. 56:965:-973; and Irizarry, et at. (1997) J. Neurosci. 17:7053-7059. A number of reports indicate that reactive oxygen species (ROS) is related to neuronal damage and degeneration in Alzheimer's disease. See Smith, et al. (1994) PNAS USA 91:5710-5714; Smith, et al. (1994) Am. J. Pathol. 145:42-47; Smith, et al. (1995) Trends Neurosci. 18:172-176; Smith, et al. (1995) J. Neurochem. 64:2660-2666; Smith, et al. (1995) Nature 374:316; Smith, et al. (1996) Nature 382:120-121; Smith, et al. (1996) Brain Res. 717:99-108; Smith, et al. (1997) J. Neurosci. 17:2653-2657; Smith, et al. (1997) PNAS USA 94:9866-9868; and Sayre, et al. (1997) J. Neurochem. 68:2092-2097. As the aging process is associated with an increase in the adventitious production of ROS together with a concurrent decrease in the ability to defend against such ROS suggests that oxidative stress may be important in the pathogenesis of Alzheimer's disease. See Harman (1956) J. Gerontol. 11:298-300.

Damage due to oxidative stress in Alzheimer's disease includes advanced glycation end products, nitration, lipid peroxidation adduction products and carbonyl-modified protein. See Ledesma, et al. (1994) J. Biol. Chem. 269:21614-21619; Vitek, et al. (1994) PNAS USA 91:4766-4770; Yan, et al. (1994) PNAS USA 91:7787-7791; Good, et al. (1996) Am. J. Pathol. 149:21-28; Montine, et al. (1996) Am. J. Pathol. 148:89-93; and Smith, et al. (1991) PNAS USA 88:10540-10543. Oxidative damage is an extremely early pathologic event as the damage extends beyond the lesions and to neurons not displaying obvious degenerative change. Oxidative damage induces the up-regulation of the antioxidant enzyme, heme oxygenase-1 in neurons with NFT. See Schipper, et al. (1995) Ann. Neurol. 37:758-768; and Premkumar, et at. (1995) J. Neurochem. 65:1399-1402.

Although increased oxidative damage is a prominent and early feature of vulnerable neurons in Alzheimer's disease and damage to proteins, sugars, lipids, nucleic acids and organelles are evident, the source of increased ROS has not been determined. The production of reactive oxygen species occurs as a ubiquitous byproduct of both oxidative phosphorylation and the myriad of oxidases necessary to support aerobic metabolism. In Alzheimer's disease, there are a number of additional contributory sources that are thought to play an important role in the disease process including: (1) iron, in a redox-active state, which is increased in neurofibrillary tangles as well as in amyloid-β deposits; (2) activated microglia, such as those that surround most senile plaques, are a source of NO and O₂⁻ which can react to form peroxynitrite, thereby leaving nitrotyrosine as an identifiable marker; (3) amyloid-β which is implicated in the formation of free radicals through peptidyl radicals; and (4) advanced glycation end products in the presence of transition metals which may undergo redox cycling with consequent production of free radicals. See Good, et al. (1992) Ann. Neurol. 31,286-292; Cras, et al. (1990) Am. J. Pathol. 137:241-246; Colton, et al. (1987) FEBS Lett. 223:284-288; Butterfield, et al. (1994) Biochem. Biophys. Res. Commun. 200:710-715; Hensley, et al. (1994) PNAS USA 91:3270-3274; Sayre, et al. (1997) Chem.Res. Toxicol. 10:518-526; Baynes, J. W. (1991) Diabetes 40:405-412; and Yan, et al. (1995) Nature Medicine 1:693-699. The advanced glycation end products and amyloid-β may activate the receptor for advanced glycation end (RAGE) products and thereby produce oxidizing species. See Yan, et al. (1996) Nature 382:685-691; and El Khoury, et al. (1996) Nature 382:716-719.

Metabolic abnormalities may also play a role in free radical formation. See Corral-Debrinski, et al. (1994) Genomics 23:471-476; Davis, et al. (1997) PNAS USA 94:4526-4531; Sorbi, et al. (11983) Ann. Neurol. 13:72-78; Sheu, et al. (1985) Ann. Neurol. 17:444-449; Sims, et al. (1987) Brain Res. 436:30-38; Blass, et al. (1990) Arch. Neurol. 47:864-869; and Parker, et al. (1990) Neurology 40:1302-1303. Neuronal damage by amyloid-β may be mediated by free radicals, which free radicals may be attenuated with antioxidants such as vitamin E or catalase. See Behl, et al. (1992) Biochem. Biophys. Res. Commun. 186:944-950; Behl, et al. (1994) Cell 77:817-827; Lockhart, et al. (1994) J. Neurosci. Res. 39:494-505; and Zhang, et al. (1996) J. Neurochem. 67:1595-1606. Presenilins 1 and 2 may also involve oxidative damage and increased presenilin 2 expression increases DNA fragmentation and apoptotic changes. See Sherrington, et al. (1995) Nature 375:754-760; and Wolozin, et al. (1996) Science 274:1710-1713. Apolipoprotein E, in brains and cerebrospinal fluid, is found adducted with the highly reactive lipid peroxidation product, hydroxynanenal. See Montine, et at (1996) J. Neuropathol. Exp. Neural. 55:202-210.

Furthermore, apolipoprotein E is a strong chelator of copper and iron, important redox-active transition metals. See Miyata, et al. (1996) Nature Genetics 14:55-61. Finally, interaction of apolipoprotein E with amyloid-β only occurs in the presence of oxygen. See Strittmatter, et al. (1993) PNAS USA 90:8098-8102.

Both free radical formation inhibition and metal chelation treatment reduce the incidence and the progression of Alzheimer's disease, thereby suggesting that oxidative stress precedes cell and tissue damage. See McGeer, et al. (1992) Neurology 42:447-449; Rogers, et al. (1993) Neurology 43:1609-1611; Breitner, et al. (1994) Neurology 44:227-232; Munch, et al. (1994) J. Neural. Trans-Parkinsons Dis. Dem. Sect. 8:193-208; Munch, et al. (1997) Biochim. Biophys. Acta 1360:17-29; Rich, et al. (1995) Neurology 45:51-55; Colaco, et at. (1996) Nephrology, Dialysis, Transplantation 11 (Suppl5):7-12; Kanowski, et al. (1996) Pharmacopsychiatry 29:47-56; Smalheiser, et al. (1996) Neurology 46:583; Stoll, et al. (1996) Pharmacopsychiatry 29: 144-149; Thal, et al. (1996) Neurology 47:705-711; Henderson, V. W. (1997) Neurology 48 (Suppl 7): S27-S35; Kawas, et al. (1997) Neurology 48:1517-1521; Papasozomenos, S. C. (1997) PNAS USA 94:6612-6617; Sano, et al. (1997) New Eng. J. Med. 336:1216-1222; Shoda, et al. (1997) Endocrinology 138:1886-1892; Skolnick, A. A. (1997) JAMA 277:776; Stewart, et al. (1997) Neurology 48:626-631; and McLachlan, et al. (1991) Can. Med. Assoc. 145:793-804.

Whether oxidative stress is a central process in neurodegeneration or instead a result of the disease process and whether it is a primary or secondary event in disease pathogenesis are important questions in determining the therapeutic value of reducing oxidative stress in Alzheimer's disease treatments. See Gotz, et al. (1994) PNAS USA 91:3270-3274; and Mattson, et al. (1995) Nature 373:481. However, efforts to elucidate the role of oxidative stress in Alzheimer's disease have been limited by the lack of a suitable cellular model as the vulnerable neurons of the brain cannot be maintained in culture. Therefore, there exists a need for an oxidative stress cellular model and a method of making and using the same.

### SUMMARY OF THE INVENTION

In some embodiments, the present invention relates to a method for detecting or measuring the amount of oxidative stress or damage in a subject suspected of having Alzheimer's disease comprising obtaining a sample from the subject, and detecting or measuring an amount of an oxidative stress marker in the sample. The sample is a neuron sample, preferably an olfactory neuron sample. The subject is mammalian, preferably human. The oxidative stress marker is carboxymethyllysine (CML), 4-hydroxy-2-nonenal (HNE), heme-oxygenase-I (HO-I), amyloid protein precursor, nitrotyrosine (NT), 8-hydroyguanosine (8OHG), pentosidine, tau, or pyrraline. Preferably, the oxidative stress marker is carboxymethyllysine (CML), 4-hydroxy-2-nonenal (HNE), heme-oxygenase-I (HO-I), amyloid protein precursor, pentosidine, or pyrraline.

In some embodiments, the invention relates to a method of screening for a candidate compound that inhibits, reduces, or prevents oxidative stress or damage comprising applying the candidate compound to a first olfactory neuron culture, detecting or measuring an oxidative stress marker in the first olfactory neuron culture to obtain a first amount, obtaining a second amount of the oxidative stress marker from a control olfactory neuron culture, and comparing the first amount to the second amount. In embodiments where the first olfactory neuron culture is under conditions of oxidative stress, then the control olfactory neuron culture is not under conditions of oxidative stress. In embodiments where the first olfactory neuron culture is obtained from a subject suspected of having Alzheimer's disease then the control olfactory neuron culture is obtained from a subject not suspected of having Alzheimer's disease.

In some embodiments, the present invention relates to a method for diagnosing Alzheimer's disease in a subject comprising obtaining an olfactory neuron sample from the subject, measuring or detecting an amount of an oxidative stress marker in the sample, and comparing the amount with a control. The subject is diagnosed with Alzheimer's disease if the amount measured or detected is the same as the control where the control is an amount determined to be characteristic of subjects having Alzheimer's disease. Alternatively, the subject is diagnosed with Alzheimer's disease if the amount measured or detected is more than the control where the control is an amount determined to be characteristic of normal subjects not afflicted with Alzheimer's disease.

In some embodiments, the present invention relates to a method of treating a subject suspected of having Alzheimer's disease comprising administering a compound, determined to reduce, inhibit, or prevent oxidative stress by the screening method of the present invention, to the subject. The compound is administered in a therapeutically effective amount and may be administered as a suitable pharmaceutical formulation.

In some embodiments, the present invention relates to a method of reducing, inhibiting, or preventing oxidative damage in a subject comprising administering a compound determined to reduce, inhibit, or prevent oxidative stress by the screening method of the invention to the subject. In preferred embodiments, the oxidative damage is neurodegeneration.

### BRIEF DESCRIPTION OF THE DRAWINGS

This invention is further understood by reference to the drawings wherein:

FIG. 1 illustrates that oxidative damage in olfactory neuron biopsy specimens exhibiting a higher level of HO-I as compared to the control.

FIG. 2 illustrates that oxidative damage in olfactory neuron biopsy specimens exhibiting a higher level of CML as compared to the control.

FIG. 3 illustrates that oxidative damage in olfactory neuron biopsy specimens exhibiting a higher level of HNE as compared to the control.

FIG. 4 illustrates that oxidative damage in olfactory neuron biopsy specimens exhibiting a higher level of pentosidine as compared to the control.

FIG. 5 illustrates that oxidative damage in olfactory neuron biopsy specimens exhibiting a higher level of amyloid protein precursor as compared to the control.

### DETAILED DESCRIPTION OF THE INVENTION

The present invention relates to methods of making and using neurons for the study and treatment of oxidative stress related disorders and diseases. In particular, the present invention relates to olfactory neurons that may be cultured and used for the study and treatment of Alzheimer's disease type oxidative stress. Olfactory neurons are preferred as olfactory neurons obtained from Alzheimer's disease subjects exhibit pathological differences from olfactory neurons obtained from normal subjects and because cultured olfactory neurons show an Alzheimer's disease related increase in the lipid peroxidation marker carboxymethyllysine (CML) and the oxidative response protein, heme oxygenase-I (HO-1). The olfactory neurons are preferably human.

As used herein, “oxidative stress” or “oxidative damage” means the consequences of free radical dependent damage to proteins, nucleic acids, or lipids without the regard to the specific radical involved or the relative preponderance of the targets. “Oxidative damage” includes neurodegeneration.

As used herein, “oxidative stress disorders and diseases” means any disorder or disease caused by oxidative stress or damage or of which oxidative stress or damage is a symptom.

As used herein, “Alzheimer's disease subject” refers to a subject diagnosed with probable Alzheimer's disease based upon National Institute of Neurological Disorders and Stroke and the Alzheimer's Disease and Related Disorders Association (NINDS-ADRDA) criteria. See McKhann, et at. (1984).

As used herein, the olfactory neurons may be from a sample of olfactory epithelium or cultured olfactory neuron cell lines such as those disclosed in Wolozin, et al. U.S. Pat. No. 5,869,266, which is herein incorporated by reference.

Cultures of human olfactory neurons may be established from tissue samples containing neurons, such as the olfactory epithelium. Samples of the olfactory epithelium are embedded in reconstituted basement membrane. The basement membrane may comprise laminin, collagen, preferably collagen IV, heparan sulfate, proteoglycans, entactin and nidogen, TGF-beta, fibroblast growth factor, tissue plasminogen activator, and other suitable growth factors such as those which occur naturally in the EHS tumor, or a combination thereof. A suitable basement membrane is commercially available from Becton Dickinson, product #354234, Bedford, Mass. Then the samples are incubated in Coon's 4506 media, a Ham's F-12 based medium for neuroblast formation, as disclosed in U.S. Pat. No. 5,910,443, which is incorporated herein by reference, which is supplemented with about 6% fetal calf serum, about 1.0 μg/ml insulin, about 40 μg to about 40 μg/ml thyroxine, about 2.5 ng/ml sodium selenite, about 60 g/ml gentamycin, about 5 μg/ml human transferrin, about 150 μg/ml bovine hypothalamus extract, about 50 μg/ml bovine pituitary extract, and about 3.5 ng/ml hydrocortisone.

As oxidative stress results in numerous deleterious cellular consequences such as lipoperoxidation, glycoxidation, protein oxidation, protein cross-linking and nucleic acid fragmentation, the oxidative damage may be analyzed with markers including carboxymethyllysine (CML); 4-hydroxy-2-nonenal (HNE), a product of lipid peroxidation; heme-oxygenase-1 (HO-1), which is induced in cells undergoing oxidative stress; amyloid protein precursor, nitrotyrosine (NT) (Upstate Biotechnology, Lake Placid, N.Y.); 8-hydroyguanosine (8OHG) (Trevigen, Gaithersburg, Md.), a marker of oxidized nucleoside present in damaged RNA and DNA; pentosidine, and pyrraline, both markers of glycation. Antisera against CML, HO-1, HNE, pentosidine and pyrraline may be made by methods standard in the art. The oxidative stress may also be analyzed by methods that localize redox-active iron, in situ hybridization of mtDNA deletion and the dinitrophenylhydrazine (DNPH) assay and other suitable oxidative stress assays known in the art.

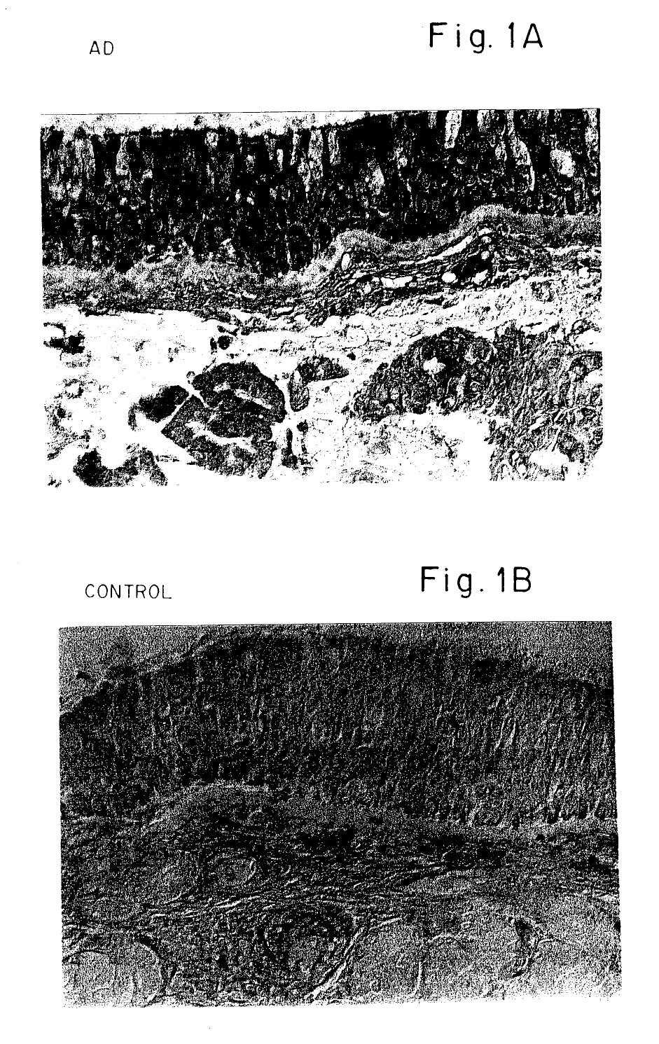

Cellular responses to ROS include up-regulation of protective responses which may be detected and measured as indicators of oxidative stress. Protective responses include the increased activity or production of heme oxygenase-I, iron regulatory proteins, and sulfhydryl reduction. As explained in Example 3, and illustrated in FIG. 1, HO-I is an excellent marker of oxidative stress in olfactory neuron cultures.

Nitrotyrosine is a marker of oxidative stress because oxidative stress is associated with high local concentrations of superoxide and nitric oxide, which are formed by the inducible isoform of nitric oxide synthase and combine to form peroxynitrite. In the presence of a bicarbonate buffer, peroxynitrite forms a CO₂ adduct, 3-nitrotyrosine. Nitrotyrosine assay controls include omitting the primary antibody, adsorption of the antibody with nitrated proteins or peptides, and chemical reduction of nitrotyrosine by sodium hydrosulfite prior to immunostaining performed in parallel with the antisera to known markers as controls against artifactual inactivation of either primary or secondary antibodies from the use of sodium hydrosulfite-reduced sections. However, as explained in Example 3, there was no change in the amount of nitrotyrosine in olfactory neuron cultures obtained from Alzheimer's disease subjects as compared to controls. Therefore, nitrotyrosine appears to not be a suitable marker of oxidative stress in olfactory neuron cultures.

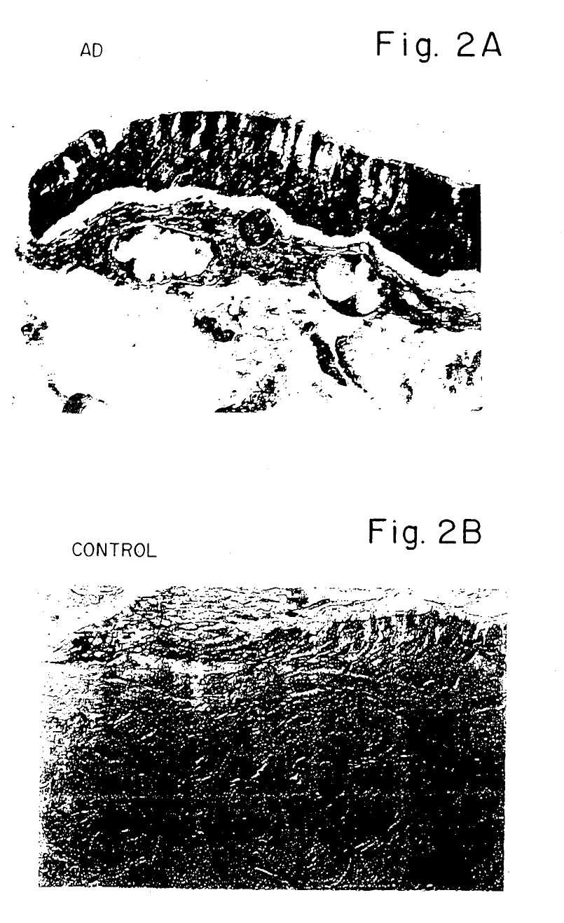

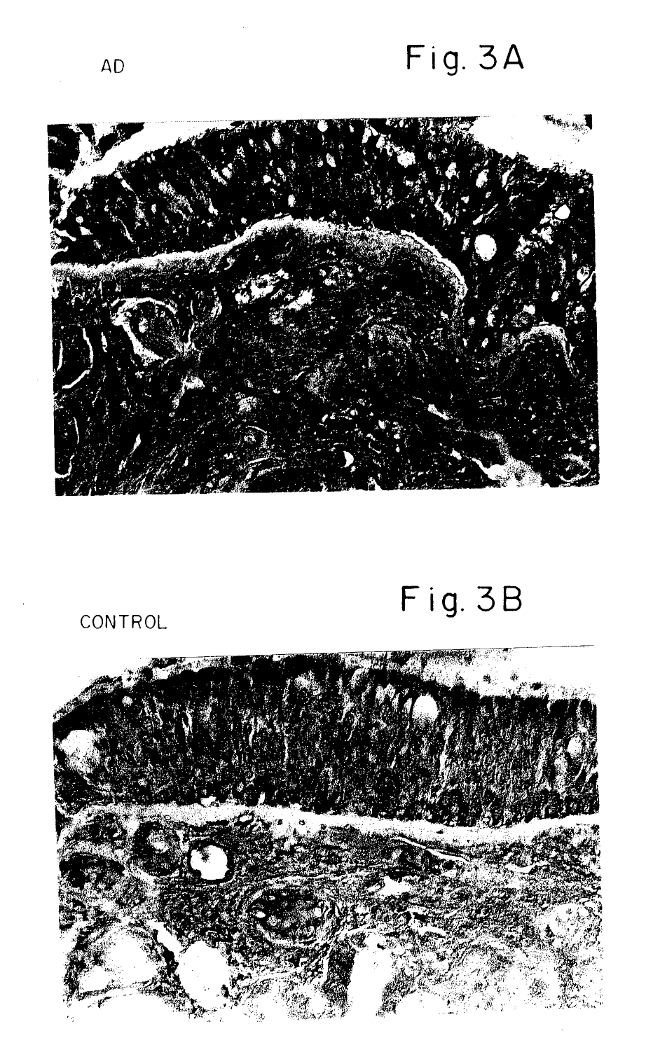

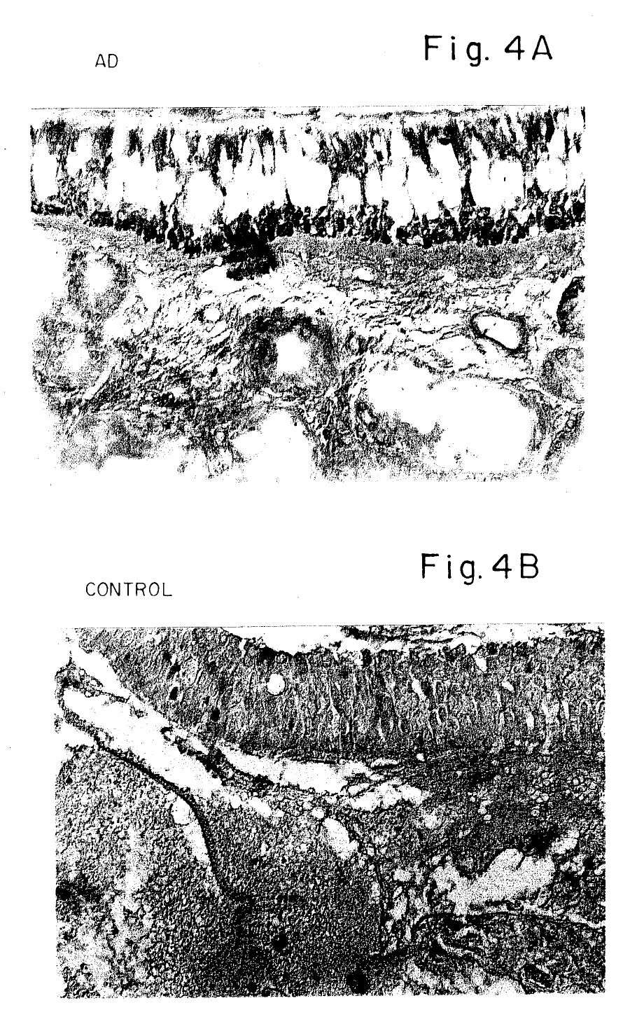

Pentosidine, pyrraline, HNE, carboxymethyllysine (CML) and malondialdehyde, are Maillard reaction products, which are markers of oxidative stress. The Maillard reaction is initiated by the nonenzymatic condensation of a reducing sugar with a protein amino group to form a Schiff base, which then undergoes an Amadori rearrangement to regenerate carbonyl reactivity. Subsequent reactions involving dehydration, rearrangement, fragmentation, and further condensation reactions yield a variety of Maillard reaction end products. As explained in Example 3 and illustrated id FIGS. 2-4, pentosidine, CML and FINE are excellent markers of oxidative stress in olfactory neuron cultures.

Oxidative damage to nucleic acids results in modifications, substitutions and deletions. 8OHG is a nucleic; acid modification characteristic of oxidative damage to nucleic acids and is prominent in Alzheimer's disease. The specificity of antibodies to 8OHG may be confirmed by comparing samples where the primary antibody was omitted or absorbed with purified 8OHG. The addition of DNase or RNase before incubation with 8OHG antibody can be used to determine the primary nucleic acid target of oxidative damage. Suitable markers of oxidative stress for use with olfactory neuron cultures include pentosidine, 8OHG, CML, HNE, HO-I, and other markers which illustrate a difference between olfactory neuron cultures obtained from Alzheimer's disease subjects as compared to olfactory neuron cultures obtained from normal subjects, including amyloid protein precursor as explained in Example 3 and illustrated in FIG. 6.

The olfactory neurons may be used for screening candidate compounds for compounds that affect the amount of oxidative damage or stress. For example, a candidate compound may be applied to a sample of olfactory neurons obtained from an Alzheimer's disease subject. The sample may be under basal conditions or under exogenous oxidative stress. A range of concentrations and amounts of the candidate compound may be applied to determine the concentration and amount of the candidate compound that reduces the amount of oxidative damage as compared to a control. Cell viability may be assessed by lactate dehydrogenase and trypan blue exclusion.

Screening a candidate compound comprises applying the candidate compound to a olfactory neuron sample under conditions of oxidative stress, detecting or measuring the amount of an oxidative stress marker, and comparing the amount of the oxidative stress marker with the amount of the oxidative stress marker in a suitable control. Where the amount of the oxidative stress marker is greater than that of the control, the candidate compound increases oxidative stress or damage. Where the amount of the oxidative stress marker is less than that of the control, the candidate compound inhibits, reduces, or prevents oxidative stress or damage.

A compound that inhibits, reduces or prevents oxidative damage as determined by the screening method of the present invention can be incorporated into a pharmaceutical composition suitable for administration. Such a composition typically comprises the compound and a pharmaceutically acceptable carrier. As used herein, “pharmaceutically acceptable carrier” includes any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the composition is contemplated. Supplementary active compounds can also be incorporated into the composition. Supplementary active compounds include antioxidants such as vitamins A, C and E.

The pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (EASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, or sodium chloride, in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the farm of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, adjuvant materials, or both can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories with conventional suppository bases such as cocoa butter and other glycerides or retention enemas for rectal delivery.

In one embodiment, the active compounds are prepared with carriers that will protect the compound against rapid elimination from the body, such as a controlled release formulations, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art.

It is especially advantageous to formulate oral or parenteral compositions in dosage unit form for ease of administration and uniformity of dosage. “Dosage unit form” as used herein refers to physically discrete units suited as unitary dosages for the subject to be treated, each unit containing a predetermined quantity of active compound calculated to produce the desired therapeutic effect in association with the required pharmaceutical carrier. The specifications for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from olfactory neuron culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from the olfactory neuron culture assays. A dose may be formulated in animal models to achieve a circulating plasma concentration range that includes the IC50, i.e., the concentration of the test compound which achieves a half-maximal inhibition of symptoms as determined in cell culture. Such information can be used to more accurately determine useful doses in humans. Levels in plasma may be measured, for example, by high performance liquid chromatography.

A therapeutically effective amount of the active compound may be determined by methods standard in the art. The skilled artisan will appreciate that certain factors may influence the dosage required to effectively treat a subject, including but not limited to the severity of the disease or disorder, previous treatments, the general health and age of the subject, and other diseases present. Moreover, treatment of a subject with a therapeutically effective amount of the active compound includes a single treatment or, preferably, may include a series of treatments. The effective dosage of the active compound used for treatment may increase or decrease over the course of a particular treatment. Changes in dosage may result and become apparent from the results of diagnostic assays as described herein.

The following examples are intended to illustrate but not to limit the invention.

### EXAMPLE 1

Procurement of Olfactory Neurons

Intra-nasal samples were obtained at biopsy from four Alzheimer's disease subjects and three normal control subjects. The control subjects were about 60 years or older. The samples were fixed in 10% formalin or Bouin's fixative. The samples were then embedded in paraffin and 6 μm sections were cut.

The samples were placed in modified L-15 transport medium comprising about 200 mg/l polyvinylpyrrolidone-360, about 0.79 mg/l glutathione, about 50 mg/l 2-mercaptoethanol, about 1% fetal bovine serum, about 200 U/ml penicillin, about 200 μg/ml streptomycin sulfate (all above agents were from Sigma or GIBCO) and about 2.5 μg/ml fungizone (Squibb). The samples were transported on ice. However, the samples were not frozen since freezing kills the tissue.

As an alternative to the modified L-15 transport medium described above, the L-15 transport medium may be modified to comprise some or all of the agents as described by Kischer et al. (1989) Cytotechnology 2:181-185.

### EXAMPLE 2

Culturing of Olfactory Neurons

The olfactory neurons were grown using the basic method described by Coon et al. (1989) PNAS USA 86:1703-1707. See also Ambesi-Impiombato et al. (1980) PNAS USA 77:3455-3459. The collected samples were cut into 1 mm×1 mm pieces and put under a reconstituted basement membrane preparation known as “Matrigel” available from Becton Dickinson, product #354234, Bedford, Mass., and kept in Coon's 4506 medium.

The concentrations of Mg++, Ca++, KCl, transferrin, insulin, hydrocortisone, sodium selenite acid, and gentamycin sulfate can, advantageously, be varied by about 10%; the concentrations of ascorbate, folic acid, hypoxanthine, thymidine, glucose, galactose, fetal bovine serum, T₃, bovine extracts and basement membrane can, advantageously, be varied by about 50%. Variations in the preferred ranges, as one skilled in the art will appreciate, may be acceptable, advantageous, or both. The optimal concentrations can readily be determined by one of ordinary skill in the art. After several weeks of culture, neurons began to grow. A variable number of tissue samples, between about 10 to about 100% grew out neurons. Neuronal cultures were selected based on the morphology of the cells. The basement membrane functioned to inhibit growth of other cell types and promote neuronal growth. The neurons were collected as described in Coon et al. (1989) PNAS USA 86: 1703-1707 and grown in cell culture dishes coated with a basement membrane. Dishes were coated with the basement membrane by spreading cold basement membrane on the dish and then leaving the dish at about 37° C. for at least about 10 to about 20 minutes. The Coon's 4506 medium was changed twice a week. Cells were not allowed to remain confluent for more than 2 days. The neurons were harvested from the dishes by treating the neuron cultures with a protease solution, Dispase (Boehringer-Mannheim, Indianapolis) for about 1 hr at about 37° C. The medium containing the detached cells was spun down at 1000 rpm for about 10 min, the supernatant was removed, and then the cells were resuspended in appropriate medium. Cells were always placed onto plates coated with basement membrane solution. For storage, the cells were in Coon's 4506 medium containing 10% dimethylsulfoxide. Cells were frozen down under liquid nitrogen. Clonal colonies of neurons were also obtained by diluting harvested neurons in Coon's 4506, growing them on basement membrane coated dishes, isolating individual colonies using cloning cylinders (BellCo) and then harvesting individual colonies as described above.

Coon's 4506 medium was required for initial growth of the neurons. Once established, the culture may be able to be maintained using other media such as Keratinocyte Growth Medium (Clonetics, San Diego) instead of the Coon's 4506.

### Example 3

Immunocytochemical Analysis

Immunocytochemistry was performed using standard peroxidase antiperoxidase methods known in the art. See Sternberger (1986) Immunocytochemistry, 3rd Ed. New York: Wiley. The sections of Example 1 were deparafinized in xylene and rehydrated through graded ethanol to TBS. Endogenous peroxidase activity was removed by incubating in 3% H₂O₂ for 30 minutes. The sections were incubated in 10% normal goat serum before the addition of primary antibodies that were incubated overnight at 4° C. After subsequent incubation in secondary antibody and peroxidase anti-peroxidase complexes, immunoreactions were detected with 3,3′-diaminobenzidine as the chromagen. The markers used were rabbit antisera against heme-oxygenase (HO-1), hydroxynonenal (HNE), nitrotyrosine (NT), carboxymethyllysine (CML), amyloid protein precursor, pentosidine, and tau. Monoclonal antibodies against 8-hydroxyguanosine (8OHG) and pyrraline were also used. Antibodies against nitrotyrosine (NT) available from Upstate Biotechnology, Lake Placid, N.Y. and 8-hydroyguanosine (8OHG) available from Trevigen, Gaithersburg, Md., may be used. However, antiserum and monoclonal antibodies for the above markers may be made by conventional methods known in the art.

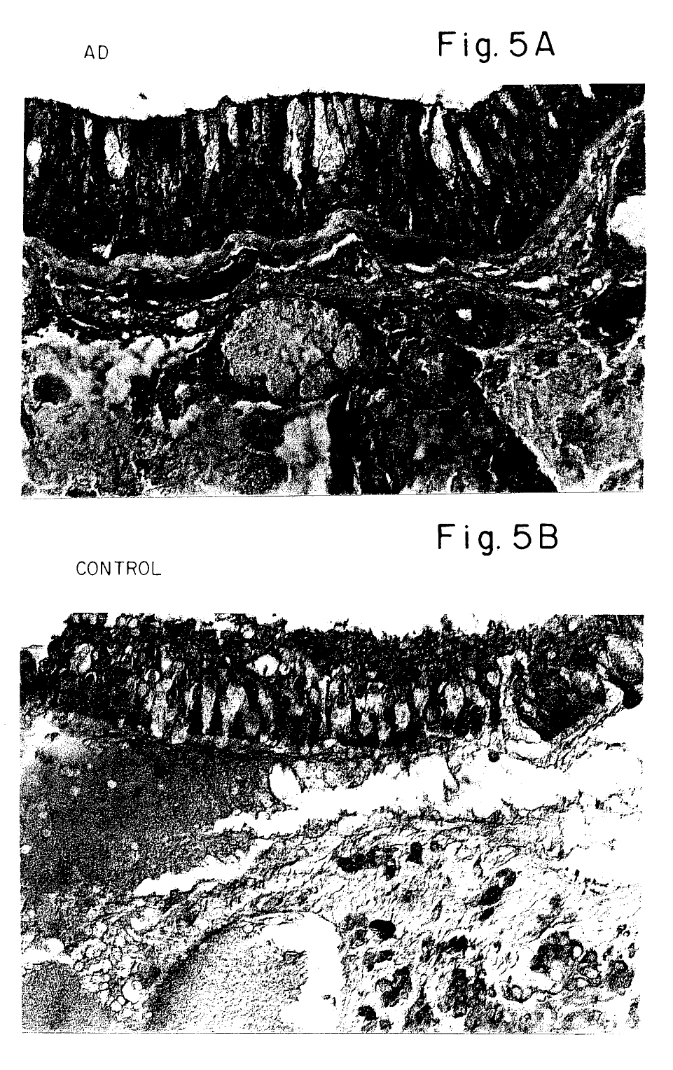

It has been previously reported that oxidative damage in neurons in Alzheimer's disease brain, obtained at autopsy, may be evidenced by markers of HO-1, HNE, NT, pentosidine, pyrraline and others. As shown in FIGS. 1-6, these same markers can be used to differentially label olfactory neurons in cases of Alzheimer's disease as compared to controls. These figures show higher levels of 8OHG, HO-1, CML, HNE, and pentosidine, as seen by a darker brown staining, in the epithelial layer of the olfactory biopsy specimens. However, it was found that antibodies against NT and tau did not provide a detectable difference between olfactory neuron samples obtained from Alzheimer's disease subjects as compared to controls (data not shown). Therefore, markers for NT and tau are not suitable for detecting or measuring oxidative stress and damage in olfactory neurons.

### EXAMPLE 4

Oxidative Stress

Oxidative stress induced by applying about 25 to about 100 μM H₂O₂ to the olfactory neuron cultures of Example 2. Alternatively, glucose and glucose oxidase may be used as sustained sources of H₂O₂.

### EXAMPLE 5

Candidate Screening

Candidate compounds may be applied to cultures of olfactory neurons obtained from either Alzheimer's disease subjects or normal subjects under basal conditions or under exogenous oxidative stress to screen for compounds which reduce, inhibit or prevent oxidative damage.

The candidate compounds may be added to the cell culture media. Oxidative stress may be induced by the addition of H₂O₂ or glucose and glucose oxidase. The cells may be examined by using the markers to oxidative damage, as explained above and in Example 3, and analyzed for the amount and types of oxidative stress products were produced. The candidate compounds that reduce, inhibit, or prevent oxidative damage may be used to confer resistance to oxidative stress caused by H₂O₂ or glucose and glucose oxidase. Preferably, the candidate compounds that reduce, inhibit, or prevent oxidative damage may be used in the treatment of cell or subjects suffering from oxidative stress and damage.

To the extent necessary to understand or complete the disclosure of the present invention, all publications, patents, and patent applications mentioned herein are expressly incorporated by reference therein to the same extent as though each were individually so incorporated.

What is claimed is:

1. A method for detecting or measuring the amount of oxidative stress or damage in a subject suspected of having Alzheimer's disease, comprising obtaining a sample from the subject, and detecting or measuring an amount of an oxidative stress marker in the sample.

2. The method of claim 1, wherein the sample is a neuron sample.

3. The method of claim 2, wherein the neuron sample is an olfactory neuron sample.

4. The method of claim 3, wherein the subject is human.

5. The method of claim 1 wherein the oxidative stress marker is carboxymethyllysine (CML), 4-hydroxy-2-nonenal (HNE), heme-oxygenase-I (HO-I), amyloid protein precursor, nitrotyrosine (NT), 8-hydroyguanosine (8OHG), pentosidine, or pyrraline.

6. The method of claim 5, wherein the oxidative stress marker is carboxymethyllysine (CML), 4-hydroxy-2-nonenal (HNE), heme-oxygenase-I (HO-I), 15 amyloid protein precursor, pentosidine, or pyrraline.

7. A method of screening for a candidate compound that modulates, inhibits, reduces, or prevents oxidative stress or damage comprising applying the candidate compound to a first olfactory neuron culture, detecting or measuring an oxidative stress marker in the first olfactory neuron culture to obtain a first amount, obtaining a second amount of the oxidative stress marker from a control olfactory neuron culture, and comparing the first amount to the second amount.

8. The method of claim 7, wherein the first olfactory neuron culture is under conditions of oxidative stress and the control olfactory neuron culture is not under conditions of oxidative stress.

9. The method of claim 7, wherein the first olfactory neuron culture is obtained from a subject suspected of having Alzheimer's disease and the control olfactory neuron culture is obtained from a subject not suspected of having Alzheimer's disease.

10. A method for diagnosing Alzheimer's disease in a subject comprising obtaining an olfactory neuron sample from the subject, measuring or detecting an amount of an oxidative stress marker in the sample, and comparing the amount with a control.

11. The method of claim 10, wherein the subject is diagnosed with Alzheimer's disease if the amount measured or detected is the same as the control where the control is an amount determined to be characteristic of subjects having Alzheimer's disease.

12. The method of claim 10, wherein the subject is diagnosed with Alzheimer's disease if the amount measured or detected is more than the control where the control is an amount determined to be characteristic of normal subjects not afflicted with Alzheimer's disease.

13. A method of treating a subject suspected of having Alzheimer's disease, comprising administering a compound determined to reduce, inhibit, or prevent oxidative stress by the method of claim 7 to the subject.

14. The method of claim 13, wherein the compound is administered in a therapeutically effective amount.

15. The method of claim 13, wherein the compound is administered as a suitable pharmaceutical formulation.

16. A method of modulating, reducing, inhibiting, or preventing oxidative damage in a subject comprising administering a compound determined to reduce, inhibit, or prevent oxidative stress by the method of claim 7 to the subject.

17. The method of claim 16, wherein the oxidative damage is neurodegeneration.
